(19) United States
(12) Patent Application Publication
(10) Pub. No.: US 2016/0272674 A1
(43) Pub. Date: Sep. 22, 2016

(54) ISOLATION AND PURIFICATION OF ANTIBODIES

(71) Applicant: ABBVIE INC., North Chicago, IL (US)

(72) Inventors: Heidi Althouse, Worcester, MA (US); Shilpa Ananthakrishnan, Worcester, MA (US); Germano Coppola, Shrewsbury, MA (US); Scott T. Ennis, South Grafton, MA (US); Robert K. Hickman, Worcester, MA (US); Chen Wang, Shrewsbury, MA (US); Joe Yakamavich, Worcester, MA (US)

(21) Appl. No.: 15/035,091

(22) PCT Filed: Nov. 7, 2014
(86) PCT No.: PCT/US14/64635

§ 371 Date: May 6, 2016

Related U.S. Application Data
(60) Provisional application No. 61/901,242, filed on Nov. 7, 2013.
(60) Provisional application No. 61/901,214, filed on Nov. 7, 2013.
(60) Provisional application No. 61/901,228, filed on Nov. 7, 2013.
(60) Provisional application No. 61/901,183, filed on Nov. 7, 2013.

(51) Int. Cl. C07K 1/16; C07K 16/00; C07K 1/36; C07K 1/22; C07K 1/34

(52) U.S. Cl. CPC C07K 1/165; C07K 1/22; C07K 1/34; C07K 1/36; C07K 16/00

(57) ABSTRACT

Embodiments of the present invention are directed to high throughput flow through purification of antibodies using mixed mode chromatography.

### STATEMENT REGARDING FEDERAL FUNDING

Embodiments of the present invention were not conceived or developed with Federal sponsorship or funding.

### BACKGROUND OF THE INVENTION

Purification processes for pharmaceutical grade monoclonal antibodies produced by fermentation culture typically involve four basic steps. These steps include (1) harvest/clarification—separation of host cells from the fermentation culture; (2) capture—separation of antibody from the majority of components in the clarified harvest; (3) fine purification—removal of residual host cell contaminants and aggregates; and (4) formulation—place the antibody into an appropriate carrier for maximum stability and shelf life.

However, these steps often do not necessarily result in antibody compositions of sufficient purity for use in pharmaceutical contexts. There is a present need for methods of producing and purifying an antibody of interest in sufficiently pure form to be suitable for pharmaceutical use. The present invention addresses this need.

### SUMMARY OF THE INVENTION

The present invention is directed to methods for isolating and purifying antibodies from a sample. In certain aspects, the invention is directed to methods of antibody purification employing affinity chromatography, preferably Protein A chromatography. In specific aspects, the methods herein employ an affinity chromatographic step, and one or more additional chromatography and/or filtration steps. The chromatography steps can include one or more steps of ion exchange and hydrophobic interaction chromatography (HIC). That is, ion exchange and hydrophobic interaction chromatography are performed concurrently, as a single step, as mixed mode chromatography with the use of mixed mode resins. Further, the present invention is directed toward pharmaceutical compositions comprising one or more antibodies purified by a method described herein.

One embodiment or the present invention is directed toward a method of purifying an antibody or antigen-binding portion thereof from a sample such that the resulting antibody composition is substantially free of process- and product-related impurities including host cell proteins (“HCPs”), leached Protein A, aggregates, and fragments. In one aspect, the sample comprises a cell line harvest wherein the cell line is employed to produce specific antibodies of the present invention.

In one embodiment, the affinity chromatography step comprises subjecting the primary recovery sample to a column comprising a suitable affinity chromatographic support. Non-limiting examples of such chromatographic supports include, but are not limited to Protein A resin, Protein G resin, affinity supports comprising the antigen against which the antibody of interest was raised, and affinity supports comprising an Fc binding protein. Protein A resin is useful for affinity purification and isolation of antibodies (IgG). In one aspect, a Protein A column is equilibrated with a suitable buffer prior to sample loading. An example of a suitable buffer is a Tris/NaCl buffer, pH around 7.2. Following this equilibration, the sample can be loaded onto the column. Following the loading of the column, the column can be washed one or multiple times using, e.g., the equilibrating buffer. Other washes including washes employing different buffers can be used before eluting the column. The Protein A column can then be eluted using an appropriate elution buffer. An example of a suitable elution buffer is an acetic acid/NaCl buffer, pH around 3.5. The eluate can be monitored using techniques well known to those skilled in the art. For example, the absorbance at OD₂₈₀ can be followed. The eluated fraction(s) of interest can then be prepared for further processing.

In one embodiment, a mixed mode step follows Protein A affinity chromatography. This mixed mode step can feature either cation or anion exchange or a combination of both. This step can be based on a single type of ion exchanger mixed mode procedure or can include multiple ion exchanger mixed mode steps such as a cation exchange mixed mode step followed by an anion exchange mixed mode step or vice versa. In one aspect, the ion exchange mixed mode step is a one-step procedure. In another aspect, the ion exchange mixed mode step involves a two-step ion exchange mixed mode process. A suitable cation exchange column is a column whose stationary phase comprises anionic groups. An example of such a column is a Capto MMC™, Capto MMC™ ImpRes (GE Healthcare), Nuvia™ cPrime™ (Biorad). In another aspect, a suitable anion exchange column is a column whose stationary phase comprises cationic groups. An example of such a column is a Capto Adhere™, and Capto Adhere™ ImpRes (GE Healthcare). One or more ion exchanger mixed mode steps further isolates antibodies by reducing impurities such as host cell proteins, aggregates, fragments and DNA and, where applicable, affinity matrix protein. This mixed mode procedure is a flow-through mode of chromatography wherein the antibodies of interest do not interact or bind to the mixed mode resin (or solid phase) to a significant extent. However, many impurities do interact with and bind to the resin.

The affinity chromatography eluate is prepared for mixed mode step by adjusting the pH and ionic strength of the sample buffer. For example, the affinity eluate can be adjusted to a pH of about 5.0 to about 7.0 and conductivity adjusted to 3-15 mS/cm and then diluted to about 10 g/L. Prior to loading the sample (the affinity eluate) onto the mixed mode column, the column can be equilibrated using a suitable buffer. An example of a suitable buffer is a Tris/NaCl buffer with a pH of about 5-7.0. Following equilibration, the column can be loaded with the affinity eluate. Following loading, the column can be washed one or multiple times with a suitable buffer. An example of a suitable buffer is the equilibration buffer itself. Flow-through collection can commence, e.g., as the absorbance (OD280) rises above about 0.2 AU. The use of mixed mode flow-through chromatography reduces the amount of aggregates and HCP. The mixed mode resin has either cationic or anionic function.

In another embodiment, the mixed mode flow-through eluate is further processed through a hydrophobic interaction chromatography (HIC) step. The HIC step is operated in flow-through mode. Impurities such as HCP, leached Protein A, and aggregates can be further reduced. In one embodiment, the mixed mode resin contains anion exchange functionality such as Capto Adhere™ resin. The Capto Adhere™ flow-through eluate is adjusted to target pH (˜7.5) and ionic strength (˜350 mM sodium citrate), and flow-through a HIC resin such as phenyl Sepharose HP column. In some other aspects, the pH inactivated and filtered Protein A eluate is flowed through a HIC resin to reduce impurities.

The purity of the antibodies of interest in the resultant sample product can be analyzed using methods well known to those skilled in the art, e.g., size-exclusion chromatography, Poros™ A HPLC Assay, HCP ELISA, Protein A ELISA, and western blot analysis.

In yet another embodiment, the invention is directed to one or more pharmaceutical compositions comprising an isolated antibody or antigen-binding portion thereof and an acceptable carrier. In another aspect, the compositions further comprise one or more pharmaceutical agents.

### BRIEF DESCRIPTIONS OF THE DRAWINGS

FIG. 1 discloses the heavy and light chain variable region sequences of a non-limiting example of an anti-Tumor Necrosis Factor-alpha (TNFα) antibody (Adalimumab).

FIG. 2 depicts the results of an assay comparing the recovery of Adalimumab monomer vs. elution pH and incubation time.

FIG. 3a depicts mAb1 flow-through pool aggregate levels as a function of resin loading.

FIG. 3b depicts mAb1 flow-through pool aggregate levels as a function of yield under the tested condition.

FIG. 4a depicts mAb3 flow-through pool aggregate levels as a function of resin loading.

FIG. 4b depicts mAb3 flow-through pool aggregate levels as a function of yield under the tested condition.

FIG. 5 depicts a flow diagram embodying features of the present invention.

### DETAILED DESCRIPTION OF THE INVENTION

The present invention is directed to methods for isolating and purifying antibodies from a sample. The chromatography steps can include one or more of the following chromatographic procedures: ion exchange chromatography, affinity chromatography, and cationic mixed mode chromatography, anionic mixed mode chromatography, and hydrophobic interaction chromatography. Further, the present invention is directed toward pharmaceutical compositions comprising one or more antibodies purified by a method described herein.

For clarity and not by way of limitation, this detailed description is divided into the following sub-portions:

1. Definitions;

2. Antibody Generation;

3. Antibody Production;

4. Antibody Purification;

5. Methods of Assaying Sample Purity;

6. Further Modifications;

7. Pharmaceutical Compositions; and

8. Antibody Uses.

### 1. DEFINITIONS

In order that the present invention may be more readily understood, certain terms are first defined.

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The phrase “human repulsive guidance molecule family member A” (abbreviated herein as hRGM A or hRGMA), as used herein refers to a glycosylphosphatidylinositol (gpi)-anchored glycoprotein with 450 amino acids, was first described as a neurite growth repellent or neurite growth inhibitor during development of topographic projections (Stahl et al. Neuron 5: 735-43, 1990; Mueller, in Molecular Basis of Axon Growth and Nerve Pattern Formation, Edited by H. Fujisawa, Japan Scientific Societies Press, 215-229, 1997). The rgm gene family encompasses three different genes, two of them, rgm a and b, are expressed in the mammalian CNS, whereas the third member, rgm c, is expressed in the periphery (Mueller et al., Philos. Trans. R. Soc. Lond. B Biol. Sci. 361: 1513-29, 2006), where it plays an important role in iron metabolism. Human RGM proteins have a sequence identity of 43%-50%; the amino acid homology of human and rat RGM A is 89%. Human RGM proteins share no significant sequence homology with any other known protein. They are proline-rich proteins containing an RGD region and have structural homology to the Von-Willebrand Factor domain and are cleaved at the N-terminal amino acid 168 by an unknown protease to yield the functionally active protein (Mueller et al., Philos. Trans. R. Soc. Lond. B Biol. Sci. 361: 1513-29, 2006).

The phrase “human tumor necrosis factor-alpha” (abbreviated herein as hTNFα or TNFα) is a multifunctional pro-inflammatory cytokine secreted predominantly by monocytes/macrophages that has effects on lipid metabolism, coagulation, insulin resistance, and endothelial function. TNFα is a soluble homotrimer of 17 kD protein subunits. A membrane-bound 26 kD precursor form of TNFα also exists. It is found in synovial cells and macrophages in tissues. Cells other than monocytes or macrophages also produce TNFα. For example, human non-monocytic tumor cell lines produce TNFα as well as CD4+ and CD8+ peripheral blood T lymphocytes and some cultured T and B cell lines produce TNFα. The nucleic acid encoding TNFα is available as GenBank Accession No. X02910 and the polypeptide sequence is available as GenBank Accession No. CAA26669. The term human TNFα is intended to include recombinant human TNFα (rh TNFα), which can be prepared by standard recombinant expression methods.

The term “human antibody” includes antibodies having variable and constant regions corresponding to human germline immunoglobulin sequences as described by Kabat et al. (See Kabat, et al. (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), e.g., in the CDRs and in particular CDR3. The mutations can be introduced using the “selective mutagenesis approach.” The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immuno-globulin sequence. In other embodiments, up to ten, up to five, up to three or up to two positions are replaced. In one embodiment, these replacements are within the CDR regions. However, the term “human antibody”, as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see, e.g., Taylor, L. D., et al. (1992) Nucl. Acids Res. 20:6287-6295, the entire teaching of which is incorporated herein by reference) or antibodies prepared, expressed, created or isolated by any other means that involves splicing of human immunoglobulin gene sequences to other DNA sequences. Such recombinant human antibodies have variable and constant regions derived from human germline immunoglobulin sequences (see, Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or back-mutation or both.

An “isolated antibody” includes an antibody that is substantially free of other antibodies having different antigenic specificities (e.g., an isolated antibody that specifically binds hRGMA is substantially free of antibodies that specifically bind antigens other than hRGMA). An isolated antibody that specifically binds hRGMA may bind RGMA molecules from other species. Moreover, an isolated antibody may be substantially free of other cellular material and/or chemicals. Suitable anti-RGMA antibodies that may be purified in the context of the instant invention are disclosed in U.S. patent application Ser. No. 12/389,927 (which is hereby incorporated by reference in its entirety). A suitable anti-TNFα antibody is Adalimumab (Abbott Laboratories).

The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

The phrase “viral reduction/inactivation”, as used herein, is intended to refer to a decrease in the number of viral particles in a particular sample (“reduction”), as well as a decrease in the activity, for example, but not limited to, the infectivity or ability to replicate, of viral particles in a particular sample (“inactivation”). Such decreases in the number and/or activity of viral particles can be on the order of about 1% to about 99%, preferably of about 20% to about 99%, more preferably of about 30% to about 99%, more preferably of about 40% to about 99%, even more preferably of about 50% to about 99%, even more preferably of about 60% to about 99%, yet more preferably of about 70% to about 99%, yet more preferably of about 80% to 99%, and yet more preferably of about 90% to about 99%. In certain non-limiting embodiments, the amount of virus, if any, in the purified antibody product is less than the ID50 (the amount of virus that will infect 50 percent of a target population) for that virus, preferably at least 10-fold less than the ID50 for that virus, more preferably at least 100-fold less than the ID50 for that virus, and still more preferably at least 1000-fold less than the ID50 for that virus.

The term “aggregates” used herein means agglomeration or oligomerization of two or more individual molecules, including but not limiting to, protein dimers, trimers, tetramers, oligomers and other high molecular weight species. Protein aggregates can be soluble or insoluble.

The term “fragments” used herein refers to any truncated protein species from the target molecule due to dissociation of peptide chain, enzymatic and/or chemical modifications.

The term “host cell proteins” (HCPs), as used herein, is intended to refer to non-target protein-related, proteinaous impurities derived from host cells.

### 2. ANTIBODY GENERATION

The term “antibody” as used in this section refers to an intact antibody or an antigen binding fragment thereof.

The antibodies of the present disclosure can be generated by a variety of techniques, including immunization of an animal with the antigen of interest followed by conventional monoclonal antibody methodologies e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

One preferred animal system for preparing hybridomas is the murine system. Hybridoma production is a very well-established procedure. Immunization protocols and techniques for isolation of immunized splenocytes for fusion are known in the art. Fusion partners (e.g., murine myeloma cells) and fusion procedures are also known.

An antibody preferably can be a human, a chimeric, or a humanized antibody. Chimeric or humanized antibodies of the present disclosure can be prepared based on the sequence of a non-human monoclonal antibody prepared as described above. DNA encoding the heavy and light chain immunoglobulins can be obtained from the non-human hybridoma of interest and engineered to contain non-murine (e.g., human) immunoglobulin sequences using standard molecular biology techniques. For example, to create a chimeric antibody, murine variable regions can be linked to human constant regions using methods known in the art (see e.g., U.S. Pat. No. 4,816,567 to Cabilly et al.). To create a humanized antibody, murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In one non-limiting embodiment, the antibodies of this disclosure are human monoclonal antibodies. Such human monoclonal antibodies directed against RGMA or TNFα can be generated using transgenic or transchromosomic mice carrying parts of the human immune system rather than the mouse system. These transgenic and transchromosomic mice include mice referred to herein as the HuMAb Mouse® (Medarex, Inc.), KM Mouse® (Medarex, Inc.), and XenoMouse® (Amgen).

Moreover, alternative transchromosomic animal systems expressing human immunoglobulin genes are available in the art and can be used to raise antibodies of the disclosure, such as anti-RGMA or anti-TNFα antibodies. For example, mice carrying both a human heavy chain transchromosome and a human light chain transchromosome, referred to as “TC mice” can be used; such mice are described in Tomizuka et al. (2000) Proc. Natl. Acad. Sci. USA 97:722727. Furthermore, cows carrying human heavy and light chain transchromosomes have been described in the art (e.g., Kuroiwa et al. (2002) Nature Biotechnology 20:889-894 and PCT application No. WO 2002/092812) and can be used to raise anti-RGMA or anti-TNFα antibodies of this disclosure.

Recombinant human antibodies of the invention, including, but not limited to, anti-RGMA or anti-TNFα antibodies or an antigen binding portion thereof, or anti-RGMA-related, or anti-TNFα-related antibodies disclosed herein can be isolated by screening of a recombinant combinatorial antibody library, e.g., a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612, the entire teachings of which are incorporated herein), examples of methods and reagents particularly amenable for use in generating and screening antibody display libraries can be found in, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Human Antibody Hybridomas 3:81-85; Huse et al. (1989) Science 246:1275-1281; McCafferty et al., Nature (1990) 348:552-554; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982; the entire teachings of which are incorporated herein.

Human monoclonal antibodies of this disclosure can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In certain embodiments, the methods of the invention include anti-RGMA or anti-TNFα antibodies and antibody portions, anti-RGMA-related or anti-TNFα-related antibodies and antibody portions, and human antibodies and antibody portions with equivalent properties to anti-RGMA or anti-TNFα antibodies, such as high affinity binding to hRGMA or hTNFα with low dissociation kinetics and high neutralizing capacity. In one aspect, the invention provides treatment with an isolated human antibody, or an antigen-binding portion thereof, that dissociates from hRGMA or hTNFα with a K_{d} of about 1×10⁻⁸ M or less and a K_{off} rate constant of 1×10⁻³ s⁻¹ or less, both determined by surface plasmon resonance. In specific non-limiting embodiments, an anti-RGMA antibody purified according to the invention competitively inhibits binding of an art-known anti-RGMA antibody under physiological conditions. In specific non-limiting embodiments, an anti-TNFα antibody purified according to the invention competitively inhibits binding of Adalimumab to TNFα under physiological conditions.

### 3. ANTIBODY PRODUCTION

To express an antibody of the invention, DNAs encoding partial or full-length light and heavy chains are inserted into one or more expression vector such that the genes are operatively linked to transcriptional and translational control sequences. (See, e.g., U.S. Pat. No. 6,914,128, the entire teaching of which is incorporated herein by reference.) In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into a separate vector or, more typically, both genes are inserted into the same expression vector. The antibody genes are inserted into an expression vector by standard methods (e.g., ligation of complementary restriction sites on the antibody gene fragment and vector, or blunt end ligation if no restriction sites are present). Prior to insertion of the antibody or antibody-related light or heavy chain sequences, the expression vector may already carry antibody constant region sequences. For example, one approach to converting the anti-RGMA or anti-TNFα antibody-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

Suitable mammalian host cells for expressing the recombinant antibodies of the invention include Chinese Hamster Ovary (CHO cells) (including dhfr-CHO cells, described in Urlaub and Chasin, (1980) PNAS USA 77:42164220, used with a DHFR selectable marker, e.g., as described in Kaufman and Sharp (1982) Mol. Biol. 159:601-621, the entire teachings of which are incorporated herein by reference), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Other examples of useful mammalian host cell lines are monkey kidney CV line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2), the entire teachings of which are incorporated herein by reference.

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce an antibody may be cultured in a variety of media. Commercially available media such as Ham's F10™ (Sigma), Minimal Essential Medium™ ((MEM), (Sigma), RPMI-1640 (Sigma), and Dulbecco's Modified Eagle's Medium™ ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells, the entire teachings of which are incorporated herein by reference. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Prior to the process of the invention, procedures for purification of antibodies from cell debris initially depend on the site of expression of the antibody. Some antibodies can be secreted directly from the cell into the surrounding growth media; others are made intracellularly. For the latter antibodies, the first step of a purification process typically involves: lysis of the cell, which can be done by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by differential centrifugation or by filtration. Where the antibody is secreted, supernatants from such expression systems are generally first concentrated using a commercially available protein concentration filter, e.g., an Amicon™ or Millipore Pellicon™ ultrafiltration unit. Where the antibody is secreted into the medium, the recombinant host cells can also be separated from the cell culture medium, e.g., by tangential flow filtration. Antibodies can be further recovered from the culture medium using the antibody purification methods of the invention.

### 4. ANTIBODY PURIFICATION

### 4.1 Antibody Purification Generally

The invention provides a method for producing a purified (or “HCP-reduced”) antibody preparation from a mixture comprising an antibody and at least one HCP. The purification process of the invention begins at the separation step when the antibody has been produced using methods described above and conventional methods in the art. Table 1 summarizes one embodiment of a purification scheme. Variations of this scheme, including, but not limited to, variations where the Protein A affinity chromatography step is omitted or the order of the fine purification steps is reversed, are envisaged and are within the scope of this invention.

TABLE 1
Purification steps with their associated purpose

| Purification step | Purpose |
|---|---|
| Primary recovery | Clarification of sample matrix |
| Affinity chromatography | Antibody capture, host cell protein and |
| | associated impurity reduction |
| Low pH inactivation | Inactivate viruses |
| Anion exchange chromatography | Removing host cell protein, DNA, virus |
| Mixed mode chromatography | Reducing aggregates, fragments, HCPs, |
| | DNA, virus, leached Protein A |
| Hydrophobic interaction | Reducing aggregates, fragments, HCPs, |
| chromatography | DNA, leached Protein A |
| Viral filtration | Removal of viruses, if present |
| Final ultrafiltration/diafiltration | Concentrate and formulate proteins |

The present invention features flow-through polishing purification using mixed mode chromatography and/or hydrophobic interaction chromatography media. The mixed mode chromatography media may comprise cation exchange and/or anion exchange functions. The present invention provides for a cationic mixed mode chromatography step in substitution for or in addition to the step of anionic exchange chromatography, anionic mixed mode chromatography, or hydrophobic interaction chromatography. Thus, moving from post-Protein A low pH viral inactivation and depth filtration to viral filtration or final ultrafiltration is the step of flow-through of ionic mixed mode chromatography.

Once a clarified solution or mixture comprising the antibody has been obtained, separation of the antibody from the other proteins produced by the cell, such as HCPs, is performed using a combination of different purification techniques, including ion exchange separation step(s) and hydrophobic interaction separation step(s). The separation steps separate mixtures of proteins on the basis of their charge, degree of hydrophobicity, or size. In one aspect of the invention, separation is performed using chromatography, including cationic, anionic, and hydrophobic interaction. Several different chromatography resins are available for each of these techniques, allowing accurate tailoring of the purification scheme to the particular protein involved. The essence of each of the separation methods is that proteins can be caused either to traverse at different rates down a column, achieving a physical separation that increases as they pass further down the column, or to adhere selectively to the separation medium, being then differentially eluted by different solvents. In some cases, the antibody is separated from impurities when the impurities specifically adhere to the column and the antibody does not, i.e., the antibody is present in the flow through.

As noted above, accurate tailoring of a purification scheme relies on consideration of the protein to be purified. In certain embodiments, the separation steps of the instant invention are employed to separate an antibody from one or more HCPs. Antibodies that can be successfully purified using the methods described herein include, but are not limited to, human IgA₁, IgA₂, IgD, IgE, IgG₁, IgG₂, IgG₃, IgG₄, and IgM antibodies. In certain embodiments, the purification strategies of the instant invention exclude the use of Protein A affinity chromatography, for example in the context of the purification of IgG₃ antibodies, as IgG₃ antibodies bind to Protein A inefficiently. Other factors that allow for specific tailoring of a purification scheme include, but are not limited to: the presence or absence of an Fc region (e.g., in the context of full length antibody as compared to an Fab fragment thereof) because Protein A binds to the Fc region; the particular germline sequences employed in generating to antibody of interest; and the amino acid composition of the antibody (e.g., the primary sequence of the antibody as well as the overall charge/hydrophobicity of the molecule). Antibodies sharing one or more characteristic can be purified using purification strategies tailored to take advantage of that characteristic.

### 4.2 Primary Recovery

The initial steps of the purification methods of the present invention involve the first phase of clarification and primary recovery of antibody from a sample.

In certain embodiments, the primary recovery will include one or more centrifugation steps to further clarify the sample and thereby aid in purifying the anti-TNFα or anti-RGMa antibodies. Centrifugation of the sample can be run at, for example, but not by way of limitation, 7,000×g to approximately 12,750×g. In the context of large scale purification, such centrifugation can occur on-line with a flow rate set to achieve, for example, but not by way of limitation, a turbidity level of 150 NTU in the resulting supernatant. Such supernatant can then be collected for further purification.

In certain embodiments, the primary recovery will include the use of one or more depth filtration steps to further clarify the sample matrix and thereby aid in purifying the antibodies of the present invention. Depth filters contain filtration media having a graded density. Such graded density allows larger particles to be trapped near the surface of the filter while smaller particles penetrate the larger open areas at the surface of the filter, only to be trapped in the smaller openings nearer to the center of the filter. In certain embodiments the depth filtration step can be a delipid depth filtration step. Although certain embodiments employ depth filtration steps only during the primary recovery phase, other embodiments employ depth filters, including delipid depth filters, during one or more additional phases of purification. Non-limiting examples of depth filters that can be used in the context of the instant invention include the Cuno™ model 30/60ZA depth filters (3M Corp.), Millistak COHC, DOHC, A1HC, B1HC, XOHC, FOHC depth filters (Millipore), and 0.45/0.2 μm Sarto-pore™ bi-layer filter cartridges.

### 4.3 Affinity Chromatography

In certain embodiments, the primary recovery sample is subjected to affinity chromatography to further purify the antibody of interest away from HCPs. In certain embodiments the chromatographic material is capable of selectively or specifically binding to the antibody of interest. Non-limiting examples of such chromatographic material include: Protein A, Protein G, chromatographic material comprising the antigen bound by the antibody of interest, and chromatographic material comprising an Fc binding protein. In specific embodiments, the affinity chromatography step involves subjecting the primary recovery sample to a column comprising a suitable Protein A resin. Protein A resin is useful for affinity purification and isolation of a variety antibody isotypes, particularly IgG₁, IgG₂, and IgG₄. Protein A is a bacterial cell wall protein that binds to mammalian IgGs primarily through their Fc regions. In its native state, Protein A has five IgG binding domains as well as other domains of unknown function.

There are several commercial sources for Protein A resin. One suitable resin is MabSelect™ SuRe from GE Healthcare. A non-limiting example of a suitable column packed with MabSelect™ SuRe is an about 1.0 cm diameter and about 21.6 cm long column (˜17 mL bed volume). This size column can be used for small scale purifications and can be compared with other columns used for scale ups. For example, a 20 cm×21 cm column whose bed volume is about 6.6 L can be used for larger purifications. Regardless of the column, the column can be packed using a suitable resin such as MabSelect™ SuRe. Other non-limiting examples of Protein A resins include ProSep Ultra Plus (EMD Millipore) and Amsphere Protein ATM resin (JSR Life Sciences).

In certain embodiments it will be advantageous to identify the dynamic binding capacity (DBC) of the Protein A resin in order to tailor the purification to the particular antibody of interest. For example, but not by way of limitation, the DBC of a MabSelect™ column can be determined either by a single flow rate load or dual-flow load strategy. The single flow rate load can be evaluated at a velocity of about 300 cm/hr throughout the entire loading period. The dual-flow rate load strategy can be determined by loading the column up to about 35 mg protein/mL resin at a linear velocity of about 300 cm/hr, then reducing the linear velocity by half to allow longer residence time for the last portion of the load.

In certain embodiments, the Protein A column can be equilibrated with a suitable buffer prior to sample loading. A non-limiting example of a suitable buffer is a Tris/NaCl buffer, pH of about 7.2. A non-limiting example of a suitable equilibration condition is 25 mM Tris, 100 mM NaCl, pH of about 7.2. Following this equilibration, the sample can be loaded onto the column. Following the loading of the column, the column can be washed one or multiple times using, e.g., the equilibrating buffer. Other washes, including washes employing different buffers, can be employed prior to eluting the column. For example, the column can be washed using one or more column volumes of 20 mM citric acid/sodium citrate, 0.5 M NaCl at pH of about 6.0. This wash can optionally be followed by one or more washes using the equilibrating buffer. The Protein A column can then be eluted using an appropriate elution buffer. A non-limiting example of a suitable elution buffer is an acetic acid/NaCl buffer, pH of about 3.5. Suitable conditions are, e.g., 0.1 M acetic acid, pH of about 3.5. The eluate can be monitored using techniques well known to those skilled in the art. For example, the absorbance at OD₂₈₀ can be followed. Column eluate can be collected starting with an initial deflection of about 0.5 AU to a reading of about 0.5 AU at the trailing edge of the elution peak. The elution fraction(s) of interest can then be prepared for further processing. For example, the collected sample can be titrated to a pH of about 5.0 using Tris (e.g., 1.0 M) at a pH of about 10. Optionally, this titrated sample can be filtered and further processed.

### 4.4 Viral Inactivation and Filtration

Following the capture chromatography step is usually a virus inactivation step. For example, any one or more of a variety of methods of viral reduction/inactivation can be used including heat inactivation (pasteurization), pH inactivation, solvent/detergent treatment, UV and γ-ray irradiation and the addition of certain chemical inactivating agents such as 13-propiolactoneor, e.g., copper phenanthroline as in U.S. Pat. No. 4,534,972, the entire teaching of which is incorporated herein by reference.

Methods of pH viral reduction/inactivation include, but are not limited to, incubating the mixture for a period of time at low pH, and subsequently neutralizing the pH and removing particulates by filtration. In certain embodiments the mixture will be incubated at a pH of between about 2 and 5, preferably at a pH of between about 3 and 4, and more preferably at a pH of about 3.5. The pH of the sample mixture may be lowered by any suitable acid including, but not limited to, citric acid, acetic acid, caprylic acid, or other suitable acids. The choice of pH level largely depends on the stability profile of the antibody product and buffer components. It is known that the quality of the target antibody during low pH virus reduction/inactivation is affected by pH and the duration of the low pH incubation. In certain embodiments the duration of the low pH incubation will be from 0.5 hr to two 2 hr, preferably 0.5 hr to 1.5 hr, and more preferably the duration will be 1 hr. Virus reduction/inactivation is dependent on these same parameters in addition to protein concentration, which may limit reduction/inactivation at high concentrations. Thus, the proper parameters of protein concentration, pH, and duration of reduction/inactivation can be selected to achieve the desired level of viral reduction/inactivation.

In those embodiments where viral reduction/inactivation is employed, the sample mixture can be adjusted, as needed, for further purification steps. For example, following low pH viral reduction/inactivation the pH of the sample mixture is typically adjusted to a more neutral pH, e.g., from about 4.5 to about 8.5, prior to continuing the purification process. Additionally, the mixture may be diluted with water for injection (WFI) or supplemented with a salt solution to obtain a desired conductivity.

The pH and conductivity adjusted mixture can be filtered sequentially through synthetic depth filters; i.e. Betapore (3M) or Profile II (Pall Corp), followed by a synthetic charged depth filter (Emphaze™ (3M)) or through traditional charged depth filters (Cuno™ model 30/60ZA depth filters (3M Corp.), Millistak COHC, DOHC, A1HC, B1HC, XOHC, or FOHC depth filters (Millipore).

### 4.5 Ion Exchange Chromatography

In certain embodiments, the instant invention provides methods for producing a HCP-reduced antibody preparation from a mixture comprising an antibody and at least one HCP by subjecting the mixture to at least one ion exchange separation step such that an eluate comprising the antibody is obtained. Ion exchange separation includes any method by which two substances are separated based on the difference in their respective ionic charges, and can employ either cationic exchange material or anionic exchange material.

The use of a cationic exchange material versus an anionic exchange material is based on the overall charge of the protein. Therefore, it is within the scope of this invention to employ an anionic exchange step prior to the use of a cationic exchange step, or a cationic exchange step prior to the use of an anionic exchange step. Furthermore, it is within the scope of this invention to employ only a cationic exchange step, only an anionic exchange step, or any serial combination of the two.

In performing the separation, the initial antibody mixture can be contacted with the ion exchange material by using any of a variety of techniques, e.g., using a batch purification technique or a chromatographic technique.

For example, in the context of batch purification, ion exchange material is prepared in, or equilibrated to, the desired starting buffer. Upon preparation, or equilibration, a slurry of the ion exchange material is obtained. The antibody solution is contacted with the slurry to adsorb the antibody to be separated to the ion exchange material. The solution comprising the HCP(s) that do not bind to the ion exchange material is separated from the slurry, e.g., by allowing the slurry to settle and removing the supernatant. The slurry can be subjected to one or more wash steps. If desired, the slurry can be contacted with a solution of higher conductivity to desorb HCPs that have bound to the ion exchange material. In order to elute bound polypeptides, the salt concentration of the buffer can be increased.

Ion exchange chromatography may also be used as an ion exchange separation technique. Ion exchange chromatography separates molecules based on differences between the overall charge of the molecules. For the purification of an antibody, the antibody must have a charge opposite to that of the functional group attached to the ion exchange material, e.g., resin, in order to bind. For example, antibodies, which generally have an overall positive charge in the buffer pH below its pI, will bind well to cation exchange material, which contain negatively charged functional groups.

In ion exchange chromatography, charged patches on the surface of the solute are attracted by opposite charges attached to a chromatography matrix, provided the ionic strength of the surrounding buffer is low. Elution is generally achieved by increasing the ionic strength (i.e., conductivity) of the buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution).

Anionic or cationic substituents may be attached to matrices in order to form anionic or cationic supports for chromatography. Non-limiting examples of anionic exchange substituents include diethylaminoethyl (DEAE), quaternary aminoethyl (QAE) and quaternary amine (Q) groups. Cationic substitutents include carboxymethyl (CM), sulfoethyl (SE), sulfopropyl (SP), phosphate (P) and sulfonate (S). Cellulose ion exchange resins such as DE23™, DE32™, DE52™, CM-23™, CM-32™, and CM-52™ are available from Whatman Ltd. Maidstone, Kent, U.K. SEPHADEX®-based and -locross-linked ion exchangers are also known. For example, DEAE-, QAE-, CM and SP-SEPHADEX® and DEAE Q CM- and S-SEPHAROSE® and SEPHAROSE® Fast Flow are all available from Pharmacia AB. Further, both DEAE and CM derivitized ethylene glycol-methacrylate copolymer such as TOYOPEARL™ DEAE-6505 or M and TOYOPEARL™ CM-650S or M are available from Toso Haas Co., Philadelphia, Pa.

A mixture comprising an antibody and impurities, e.g., HCP(s), is loaded onto an ion exchange column, such as a cation exchange column. For example, but not by way of limitation, the mixture can be loaded at a load of about 80 g protein/L resin depending upon the column used. An example of a suitable cation exchange column is a 80 cm diameter×23 cm long column whose bed volume is about 116 L. The mixture loaded onto this cation column can subsequently washed with wash buffer (equilibration buffer). The antibody is then eluted from the column, and a first eluate is obtained.

This ion exchange step facilitates the capture of the antibody of interest while reducing impurities such as HCPs. In certain aspects, the ion exchange column is a cation exchange column. For example, but not by way of limitation, a suitable resin for such a cation exchange column is CM HyperDF resin. These resins are available from commercial sources such as Pall Corporation. This cation exchange procedure can be carried out at or around room temperature. This ion exchange step may also be combined with a hydrophobic interaction chromatographic process performed with resins having an ion exchange function and a hydrophobic interaction function.

### 4.6 Mixed Mode Chromatography

In certain embodiments, the pH treated and filtered Protein A eluate is further polished through one or two mixed mode chromatography columns. The mixed mode resins may contain cation exchange and hydrophobic interaction functions, or anion exchange and hydrophobic interactions. Non-limiting examples of cation exchange mixed mode resins include Capto MMC™, Capto MMC™ ImpRes (GE Healthcare, UK), Nuvia™ cPrime™ (Biorad, CA), Toyopearl MX Trp-650M (Tosoh Bioscience), while anion exchange mixed mode resins include Capto Adhere™ and Capto Adhere™ ImpRes (GE Healthcare, UK). The mixed mode column is equilibrated with a proper buffer such Tris buffer at pH 7 and conductivity about 3-20 mS/cm followed by loading of antibody feed that was pre-adjusted to similar pH and conductivity of the equilibration buffer. The column flow-through eluate is collected upon the OD₂₈₀ absorbance reaches certain threshold (e.g. 0.2 AU). The resin can be loaded to up to 1200 g/L proteins. After the feed load, the column is washed with the equilibration buffer and the wash eluate is also collected according to the OD₂₈₀ criteria.

The cation exchange mixed mode and the anion exchange mixed mode columns can be run in flow-through purification as a separate step, or together in tandem mode. The same buffers can be used for both polishing operations. The order of the two column steps can be reversed.

### 4.7 Hydrophobic Interaction Chromatography

The present invention also features methods for producing a HCP-reduced antibody preparation from a mixture comprising an antibody and at least one HCP further comprising a hydrophobic interaction separation step. For example, a first eluate obtained from an ion exchange column can be subjected to a hydrophobic interaction material such that a second eluate having a reduced level of HCP is obtained. Hydrophobic interaction chromatography steps, such as those disclosed herein, are generally performed to remove protein aggregates, such as antibody aggregates, and process-related impurities. Hydrophobic interaction chromatography steps can be performed simultaneously with ion exchange chromatography steps with chromatography resin having both ion exchange functions and hydrophobic functions. Such resins are characterized as mixed mode chromatography resins.

In performing the separation, the sample mixture is contacted with the HIC material, e.g., using a batch purification technique or using a column. Prior to HIC purification it may be desirable to remove any chaotropic agents or very hydrophobic substances, e.g., by passing the mixture through a pre-column.

For example, in the context of batch purification, HIC material is prepared in or equilibrated to the desired equilibration buffer. A slurry of the HIC material is obtained. The antibody solution is contacted with the slurry to adsorb the antibody to be separated to the HIC material. The solution comprising the HCPs that do not bind to the HIC material is separated from the slurry, e.g., by allowing the slurry to settle and removing the supernatant. The slurry can be subjected to one or more washing steps. If desired, the slurry can be contacted with a solution of lower conductivity to desorb antibodies that have bound to the HIC material. In order to elute bound antibodies, the salt concentration can be decreased.

Whereas ion exchange chromatography relies on the charges of the antibodies to isolate them, hydrophobic interaction chromatography uses the hydrophobic properties of the antibodies. Hydrophobic groups on the antibody interact with hydrophobic groups on the column. The more hydrophobic a protein is the stronger it will interact with the column. Thus the HIC step removes host cell derived impurities (e.g., DNA and other high and low molecular weight product-related species).

Hydrophobic interactions are strongest at high ionic strength, therefore, this form of separation is conveniently performed following salt precipitations or ion exchange procedures. Adsorption of the antibody to a HIC column is favored by high salt concentrations, but the actual concentrations can vary over a wide range depending on the nature of the antibody and the particular HIC ligand chosen. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba++; Ca++; Mg++; Li+; Cs+; Na+; K+; Rb+; NH₄+, while anions may be ranked in terms of increasing chaotropic effect as PO—; SO₄—; CH₃CO₃—; Cl—; Br—; NO₃—; Cl0₄-; I—; SCN—.

In general, Na, K or NH₄ sulfates effectively promote ligand-protein interaction in HIC. Salts may be formulated that influence the strength of the interaction as given by the following relationship: (NH₄)₂SO₄>Na₂SO₄>NaCl>NH₄Cl>NaBr>NaSCN. In general, salt concentrations of between about 0.75 and about 2 M ammonium sulfate or between about 1 and 4 M NaCl are useful.

HIC columns normally comprise a base matrix (e.g., cross-linked agarose or synthetic copolymer material) to which hydrophobic ligands (e.g., alkyl or aryl groups) are coupled. A suitable HIC column comprises an agarose resin substituted with phenyl groups (e.g., a Phenyl Sepharose™ column). Many HIC columns are available commercially. Examples include, but are not limited to, Phenyl Sepharose™ 6 Fast Flow column with low or high substitution (Pharmacia LKB Biotechnology, AB, Sweden); Phenyl Sepharose™ High Performance column (Pharmacia LKB Biotechnology, AB, Sweden); Octyl Sepharose™ High Performance column (Pharmacia LKB Biotechnology, AB, Sweden); Fractogel™ EMD Propyl or Fractogel™ EMD Phenyl columns (E. Merck, Germany); Macro-Prep™ Methyl or Macro-Prep™ t-Butyl Supports (Bio-Rad, California); WP HI-Propyl (C3)™ column (J. T. Baker, New Jersey); and Toyopearl™ ether, phenyl or butyl columns (TosoHaas, PA). Hydrophobic interaction resins that feature cationic functions are available commercially and include, but are not limited to, Capto MMC™, Capto MMC™ ImpRes (GE Healthcare, UK), Nuvia™ cPrime™ (Biorad, CA). Hydrophobic interaction resins (and membrane products) that feature anionic functions are available commercially and include, but are not limited to, QyuSpeed D (QSD) membrane adsorber (Ashi Kasei, Japan) and Sartobind Q membrane absorber (Sartorious AG, Germany).

### 4.8 Viral Filtration

In certain embodiments viral reduction can be achieved via the use of suitable filters. A non-limiting example of a suitable filter is the Ultipor DV20™ filter from Pall Corporation. Although certain embodiments of the present invention employ such filtration during the primary recovery phase, in other embodiments it is employed at other phases of the purification process, including as either the penultimate or final step of purification. In certain embodiments, alternative filters are employed for viral reduction, such as, but not limited to, Viresolve™ filters (Millipore, Billerica, Mass.); Virosart filter (Sartorius), Zeta Plus VR™ filters (CUNO; Meriden, Conn.); and Planova™ filters (Asahi Kasei Pharma, Planova Division, Buffalo Grove, Ill.).

### 4.9 Ultrafiltration/Diafiltration

Certain embodiments of the present invention employ ultrafiltration and/or diafiltration steps to further purify and concentrate the antibody sample. Ultrafiltration is described in detail in: Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). A preferred filtration process is Tangential Flow Filtration as described in the Millipore catalogue entitled “Pharmaceutical Process Filtration Catalogue” pp. 177-202 (Bedford, Mass., 1995/96). Ultrafiltration is generally considered to mean filtration using filters with a pore size of smaller than 0.1 nm. By employing filters having such small pore size, the volume of the sample can be reduced through permeation of the sample buffer through the filter while antibodies are retained behind the filter.

Diafiltration is a method of using ultrafilters to remove and exchange salts, sugars, and non-aqueous solvents, to separate free from bound species, to remove low molecular-weight material, and/or to cause the rapid change of ionic and/or pH environments. Microsolutes are removed most efficiently by adding solvent to the solution being ultra-filtered at a rate approximately equal to the ultrafiltration rate. This washes microspecies from the solution at a constant volume, effectively purifying the retained antibody. In certain embodiments of the present invention, a diafiltration step is employed to exchange the various buffers used in connection with the instant invention, optionally prior to further chromatography or other purification steps, as well as to remove impurities from the antibody preparations.

### 4.10 Exemplary Purification Strategies

The present invention is directed to methods for isolating and purifying antibodies from a sample. In specific aspects, the methods herein employ an affinity chromatographic step, and one or more additional chromatography and/or filtration steps. The chromatography steps can include one or more steps of ion exchange and hydrophobic interaction chromatography (HIC). That is, ion exchange and hydrophobic interaction chromatography are performed concurrently, as a single step, as mixed mode chromatography with the use of mixed mode resins. Further, the present invention is directed toward pharmaceutical compositions comprising one or more antibodies purified by a method described herein.

One embodiment or the present invention is directed toward a method of purifying an antibody or antigen-binding portion thereof from a sample such that the resulting antibody composition is substantially free of process- and product-related impurities including host cell proteins (“HCPs”), leached Protein A, aggregates, and fragments. In one aspect, the sample comprises a cell line harvest wherein the cell line is employed to produce specific antibodies of the present invention.

In one embodiment, the affinity chromatography step comprises subjecting the primary recovery sample to a column comprising a suitable affinity chromatographic support. Non-limiting examples of such chromatographic supports include, but are not limited to Protein A resin, Protein G resin, affinity supports comprising the antigen against which the antibody of interest was raised, and affinity supports comprising an Fc binding protein. Protein A resin is useful for affinity purification and isolation of antibodies (IgG). In certain aspects, the Protein A chromatography resin is selected from ProSep Ultra Plus Protein A, MabSelect SuRe™ Protein A, and Amsphere Protein ATM resins. In one aspect, a Protein A column is equilibrated with a suitable buffer prior to sample loading. An example of a suitable buffer is a Tris/NaCl buffer, pH around 7.2. Following this equilibration, the sample can be loaded onto the column. Following the loading of the column, the column can be washed one or multiple times using, e.g., the equilibrating buffer. Other washes including washes employing different buffers can be used before eluting the column. The Protein A column can then be eluted using an appropriate elution buffer. An example of a suitable elution buffer is an acetic acid/NaCl buffer, pH around 3.5. The eluate can be monitored using techniques well known to those skilled in the art. For example, the absorbance at OD²⁸⁰ can be followed. The eluated fraction(s) of interest can then be prepared for further processing.

In one embodiment, a mixed mode step follows Protein A affinity chromatography. This mixed mode step can feature either cation or anion exchange or a combination of both. This step can be based on a single type of ion exchanger mixed mode procedure or can include multiple ion exchanger mixed mode steps such as a cation exchange mixed mode step followed by an anion exchange mixed mode step or vice versa. In one aspect, the ion exchange mixed mode step is a one-step procedure. In another aspect, the ion exchange mixed mode step involves a two-step ion exchange mixed mode process. A suitable cation exchange column is a column whose stationary phase comprises anionic groups. An example of such a column is a Capto MMC™, Capto MMC™ ImpRes (GE Healthcare), Nuvia™ cPrime™ (Biorad). In another aspect, a suitable anion exchange column is a column whose stationary phase comprises cationic groups. An example of such a column is a Capto Adhere™, and Capto Adhere™ ImpRes (GE Healthcare). One or more ion exchanger mixed mode steps further isolates antibodies by reducing impurities such as host cell proteins, aggregates, fragments and DNA and, where applicable, affinity matrix protein. This mixed mode procedure is a flow-through mode of chromatography wherein the antibodies of interest do not interact or bind to the mixed mode resin (or solid phase) to a significant extent. However, many impurities do interact with and bind to the resin.

The affinity chromatography eluate is prepared for mixed mode step by adjusting the pH and ionic strength of the sample buffer. For example, the affinity eluate can be adjusted to a pH of about 5.0 to about 7.0 and conductivity adjusted to 3-15 mS/cm and then diluted to about 10 g/L. Prior to loading the sample (the affinity eluate) onto the mixed mode column, the column can be equilibrated using a suitable buffer. An example of a suitable buffer is a Tris/NaCl buffer with a pH of about 5-7.0. Following equilibration, the column can be loaded with the affinity eluate. Following loading, the column can be washed one or multiple times with a suitable buffer. An example of a suitable buffer is the equilibration buffer itself. Flow-through collection can commence, e.g., as the absorbance (OD₂₈₀) rises above about 0.2 AU. The use of mixed mode flow-through chromatography reduces the amount of aggregates and HCP. The mixed mode resin has either cationic or anionic function.

In another embodiment, the mixed mode flow-through eluate is further processed through a hydrophobic interaction chromatography (HIC) step. The HIC step is operated in flow-through mode. Impurities such as HCP, leached Protein A, and aggregates can be further reduced. In one embodiment, the mixed mode resin contains anion exchange functionality such as Capto Adhere™ resin. The Capto Adhere™ flow-through eluate is adjusted to target pH (˜7.5) and ionic strength (˜350 mM sodium citrate), and flow-through a HIC resin such as phenyl Sepharose HP column. In some other aspects, the pH inactivated and filtered Protein A eluate is flowed through a HIC resin to reduce impurities.

The purity of the antibodies of interest in the resultant sample product can be analyzed using methods well known to those skilled in the art, e.g., size-exclusion chromatography, Poros™ A HPLC Assay, HCP ELISA, Protein A ELISA, and western blot analysis.

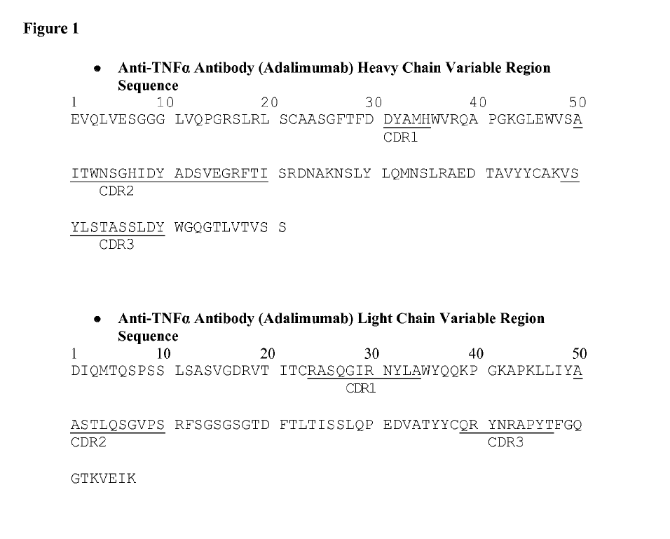

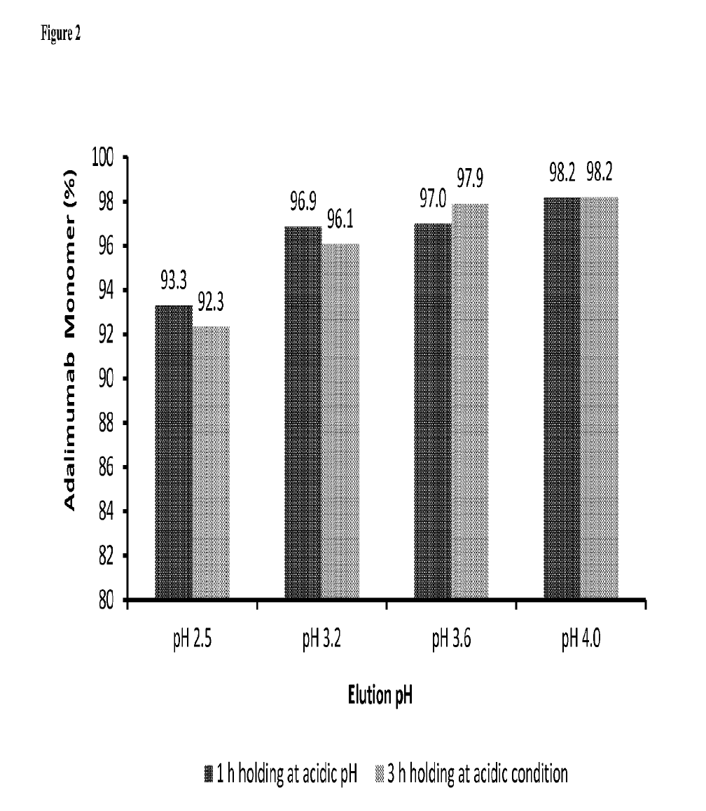

In certain embodiments of the present invention, the anti-RGMA antibody is an IgA₁, IgA₂, IgD, IgE, IgG₁, IgG₂, IgG₃, IgG₄, or IgM isotype antibody comprising heavy and light chain variable regions. In preferred embodiments, the anti-RGMA antibody is an IgG₁, IgG₂, IgG₃ or IgG₄ isotype antibody comprising heavy and light chain variable regions. More preferably the anti-RGMA antibody is an IgG₁ antibody comprising heavy and light chain variable region sequences. In certain embodiments of the present invention, the anti-TNFα antibody is an IgAⱼ, IgA₂, IgD, IgE, IgG₁, IgG₂, IgG₃, IgG₄, or IgM isotype antibody comprising the heavy and light chain variable region sequences outlined in FIG. 1. In preferred embodiments, the anti-TNFα antibody is an IgG₁, IgG₂, IgG₃ or IgG₄ isotype antibody comprising the heavy and light chain variable region sequences outlined in FIG. 1. More preferably the anti-TNFα antibody is an IgG, antibody comprising the heavy and light chain variable region sequences outlined in FIG. 1.

### 5. METHODS OF ASSAYING SAMPLE PURITY

### 5.1 Assaying Host Cell Protein

The present invention also provides methods for determining the residual levels of host cell protein (HCP) concentration in the isolated/purified antibody composition. As described above, HCPs are desirably excluded from the final target substance product, e.g., the anti-RGMA or anti-TNFα antibody. Exemplary HCPs include proteins originating from the source of the antibody production. Failure to identify and sufficiently remove HCPs from the target antibody may lead to reduced efficacy and/or adverse subject reactions.

As used herein, the term “HCP ELISA” refers to an ELISA where the second antibody used in the assay is specific to the HCPs produced from cells, e.g., CHO cells, used to generate the antibody (e.g., anti-RGMA or anti-TNFα antibody). The second antibody may be produced according to conventional methods known to those of skill in the art. For example, the second antibody may be produced using HCPs obtained by sham production and purification runs, i.e., the same cell line used to produce the antibody of interest is used, but the cell line is not transfected with antibody DNA. In an exemplary embodiment, the second antibody is produced using HPCs similar to those expressed in the cell expression system of choice, i.e., the cell expression system used to produce the target antibody.

Generally, HCP ELISA comprises sandwiching a liquid sample comprising HCPs between two layers of antibodies, i.e., a first antibody and a second antibody. The sample is incubated during which time the HCPs in the sample are captured by the first antibody, for example, but not limited to goat anti-CHO, affinity purified (Cygnus). A labeled second antibody, or blend of antibodies, specific to the HCPs produced from the cells used to generate the antibody, e.g., anti-CHO HCP Biotinylated, is added, and binds to the HCPs within the sample. In certain embodiments the first and second antibodies are polyclonal antibodies. In certain aspects the first and second antibodies are blends of poly-clonal antibodies raised against HCPs, for example, but not limited to Biotinylated goat anti Host Cell Protein Mixture 599/626/748. The amount of HCP contained in the sample is determined using the appropriate test based on the label of the second antibody.

HCP ELISA may be used for determining the level of HCPs in an antibody composition, such as an eluate or flow-through obtained using the process described above. The present invention also provides a composition comprising an antibody, wherein the composition has no detectable level of HCPs as determined by an HCP Enzyme Linked Immunosorbent Assay (“ELISA”).

### 5.2 Assaying Affinity Chromatographic Material

In certain embodiments, the present invention also provides methods for determining the residual levels of affinity chromatographic material in the isolated/purified antibody composition. In certain contexts such material leaches into the antibody composition during the purification process. In certain embodiments, an assay for identifying the concentration of Protein A in the isolated/purified antibody composition is employed. As used herein, the term “Protein A ELISA” refers to an ELISA where the second antibody used in the assay is specific to the Protein A employed to purify the antibody of interest, e.g., an anti-RGMA or anti-TNFα antibody. The second antibody may be produced according to conventional methods known to those of skill in the art. For example, the second antibody may be produced using naturally occurring or recombinant Protein A in the context of conventional methods for antibody generation and production.

Generally, Protein A ELISA comprises sandwiching a liquid sample comprising Protein A (or possibly containing Protein A) between two layers of anti-Protein A antibodies, i.e., a first anti-Protein A antibody and a second anti-Protein A antibody. The sample is exposed to a first layer of anti-Protein A antibody, for example, but not limited to polyclonal antibodies or blends of polyclonal antibodies, and incubated for a time sufficient for Protein A in the sample to be captured by the first antibody. A labeled second antibody, for example, but not limited to polyclonal antibodies or blends of polyclonal antibodies, specific to the Protein A is then added, and binds to the captured Protein A within the sample. Additional non-limiting examples of anti-Protein A antibodies useful in the context of the instant invention include chicken anti-Protein A and biotinylated anti-Protein A antibodies. The amount of Protein A contained in the sample is determined using the appropriate test based on the label of the second antibody. Similar assays can be employed to identify the concentration of alternative affinity chromatographic materials.

Protein A ELISA may be used for determining the level of Protein A in an antibody composition, such as an eluate or flow-through obtained using the process described in above. The present invention also provides a composition comprising an antibody, wherein the composition has no detectable level of Protein A as determined by a Protein A Enzyme Linked Immunosorbent Assay (“ELISA”).

### 6. FURTHER MODIFICATIONS

The antibodies of the present invention can be modified. In some embodiments, the antibodies or antigen-binding fragments thereof are chemically modified to provide a desired effect. For example, pegylation of antibodies or antibody fragments of the invention may be carried out by any of the pegylation reactions known in the art, as described, e.g., in the following references: Focus on Growth Factors 3:4-10 (1992); EP 0 154 316; and EP 0 401 384, each of which is incorporated by reference herein in its entirety. In one aspect, the pegylation is carried out via an acylation reaction or an alkylation reaction with a reactive polyethylene glycol molecule (or an analogous reactive water-soluble polymer). A suitable water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl C10) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under suitable conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments specific for RGMA or TNFα may generally be used to treat RGMA-related or TNFα-related disorders of the invention by administration of the anti-RGMA or anti-TNFα antibodies and antibody fragments described herein. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

An antibody or antigen binding portion of the invention can be derivatized or linked to another functional molecule (e.g., another peptide or protein). Accordingly, the antibodies and antigen binding portions of the invention are intended to include derivatized and otherwise modified forms of the human anti-hRGMA or anti-TNFα antibodies described herein, including immunoadhesion molecules. For example, an antibody or antigen binding portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antigen binding portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antigen binding portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

### 7. PHARMACEUTICAL COMPOSITIONS

The antibodies and antibody-portions of the invention can be incorporated into pharmaceutical compositions suitable for administration to a subject. Typically, the pharmaceutical composition comprises an antibody or antigen binding portion of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is desirable to include isotonic agents, e.g., sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antigen binding portion.

It should further be understood that the combinations which are to be included within this invention are those combinations useful for their intended purpose. The agents set forth below are illustrative and not intended to be limited. The combinations which are part of this invention can be the antibodies of the present invention and at least one additional agent selected from the lists below. The combination can also include more than one additional agent, e.g., two or three additional agents if the combination is such that the formed composition can perform its intended function.

### 8. USES OF THE ANTIBODIES OF THE INVENTION

### 8.1 Use of Anti-TNFα Antibody Generally

Tumor necrosis factor-alpha (TNFα) is a multifunctional pro-inflammatory cytokine secreted predominantly by monocytes/macrophages that has effects on lipid metabolism, coagulation, insulin resistance, and endothelial function. TNFα is a soluble homotrimer of 17 kD protein subunits. A membrane-bound 26 kD precursor form of TNFα also exists. It is found in synovial cells and macrophages in tissues. Cells other than monocytes or macrophages also produce TNFα. For example, human non-monocytic tumor cell lines produce TNFα as well as CD4′ and CDS' peripheral blood T lymphocytes and some cultured T and B cell lines produce TNFα. It is involved in, but not unique to, rheumatoid arthritis, and occurs in many inflammatory diseases. Receptors for TNFα are on several mononuclear cells, in the synovial membrane, as well as the peripheral blood and synovial fluid. TNFα is a critical inflammatory mediator in rheumatoid arthritis, and may therefore be a useful target for specific immunotherapy.

### 8.2 Use of Anti-RGMA Antibody Generally

The rgm gene family encompasses three different genes, two of them, rgm a and b, are expressed in the mammalian CNS originating RGM A and RGM B proteins, whereas the third member, RGM C, is expressed in the periphery (Mueller et al., Philos. Trans. R. Soc. Lond. B Biol. Sci. 361: 1513-29, 2006), where RGM C plays an important role in iron metabolism. In vitro, RGM A inhibits neurite outgrowth by binding to Neogenin, which has been identified as an RGM receptor (Rajagopalan et al. Nat Cell Biol.: 6(8), 756-62, 2004). Neogenin had first been described as a netrin-binding protein (Keino-Masu et al. Cell, 87(2):175-85, 1996). This is an important finding because binding of Netrin-1 to Neogenin or to its closely related receptor DCC (deleted in colorectal cancer) has been reported to stimulate rather than to inhibit neurite growth (Braisted et al. J. Neurosci. 20: 5792-801, 2000). Blocking RGM A therefore releases the RGM-mediated growth inhibition by enabling Neogenin to bind its neurite growth-stimulating ligand Netrin. Based on these observations, neutralizing RGM A can be assumed to be superior to neutralizing neogenin in models of human spinal cord injury. Besides binding of RGM A to Neogenin and inducing neurite growth inhibition, the binding of RGM A or B to the bone morphogenetic proteins BMP-2 and BMP-4 could represent another obstacle to successful neuroregeneration and functional recovery (Mueller et al., Philos. Trans. R. Soc. Lond. B Biol. Sci. 361: 1513-29, 2006).

### Examples

### 1. Flow-Through Polishing of mAb1 by Capto MMC ImpRes Resin

An Example Antibody, sometimes referred herein as mAb1, was purified by Capto MMC™ ImpRes flow-through polishing. The load material for this study was generated from a process using ProSep Ultra Plus Protein A capture followed by Mustang Q membrane polishing, and was conditioned to pH 5 and 13 mS/cm using acetic acid and 5M NaCl solution. This feed contained 1.7% of aggregates with protein concentration ˜9.3 g/L. A 1-mL HiTrap Capto MMC™ ImpRes column was used in this experiment. After equilibration the column was loaded with the respective feed up to 927 g/L at 3 min RT followed by a 20 CV of equilibration buffer wash. The flow-through and wash fractions were collected and analyzed for protein concentrations and aggregate levels.

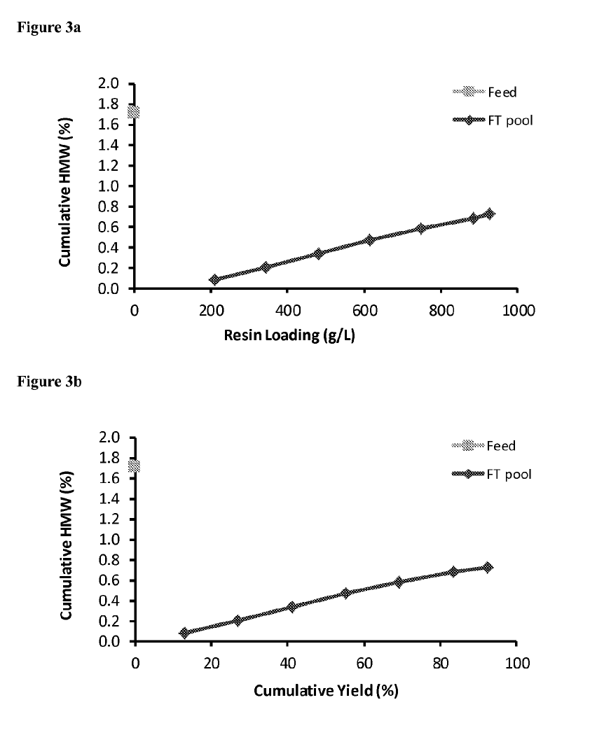

FIG. 3 shows MAB1 flow-through pool aggregate levels as a function of resin loading (a) or yield (b) under the tested condition. Without further optimization, the MAB1 aggregate level was readily reduced to ˜0.7% with 92% product recovery. Table 2 summarizes the overall process operation and performances.

TABLE 2
mAb1 purification by Protein A → Q membrane FT → Capto MMC ™ ImpRes
FT process

| | | Yield | HMW | Monomer | LMW | HCP |
|---|---|---|---|---|---|---|
| Step | Loading Conditions | (%) | (%) | (%) | (%) | (ng/mg) |
| ProSep Ultra Plus | Clarified harvest, | 93 | 2.1 | 97.3 | 0.6 | 144 |
| Protein A Capture | 1.36 g/L | | | | | |
| X0HC Filtration/ | pH 6.5, ~3.5 mS/cm, | 94 | 1.72 | 97.47 | 0.81 | 2.78 |
| Mustang Q | 0.68 kg/m² (X0HC), | | | | | |
| membrane Flow- | 2.7 kg/L Q | | | | | |
| through | membrane | | | | | |
| Capto MMC ImpRes | pH 5, 13 mS/cm, | 92 | 0.74 | 98.41 | 0.86 | 0.17 |
| Flow-through | 927 g/L | | | | | |

### 2. Flow-Through Polishing of Example mAb3 by Capto MMC™ ImpRes Resin

Capto MMC™ ImpRes flow-through method was also applied to purify a second Example Antibody, sometimes referred to herein as mAb3, in-process samples. A mAb3 MabSelect SuRe™ Protein A eluate was conditioned to pH 7 and 16.6 mS/cm, and used as the load material for Capto MMC™ ImpRes column. This feed contained 1.43% of aggregates with protein concentration ˜9.4 g/L. A 1 mL HiTrap Capto MMC™ ImpRes column was used here. After equilibration the column was loaded with the respective feed up to ˜800 g/L at 3 min RT followed by a 20 CV of equilibration buffer wash. The flow-through and wash fractions were collected and analyzed for protein concentrations and aggregate levels.

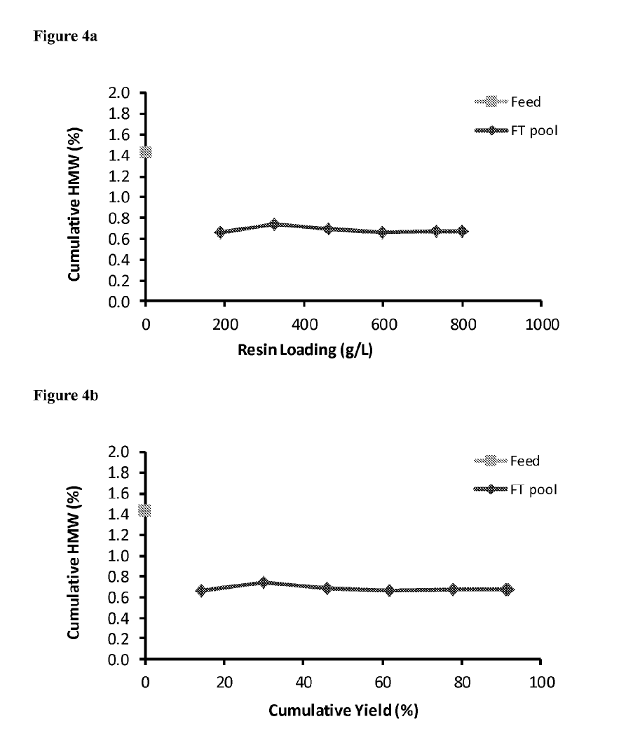

FIG. 4 shows mab3 flow-through pool aggregate levels as a function of resin loading (a) or yield (b) under the tested condition. The mAb3 aggregate level was reduced to 0.69% with 92% product recovery.

### 3. Flow-Through Polishing of mAb3 by Capto™ Adhere or Capto MMC™ Resin

Mab3, which was generated from a process using Amsphere Protein A™ resin (JSR Life Sciences) for capture followed by anion exchange depth filter polishing, was used as the load material for mixed mode resin flow-through processing. The feed was pH 7.8 and the conductivity adjusted to the targeted values (3-7.8 mS/cm). A 1 ml HiTrap Capto™ Adhere or Capto MMC™ column was equilibrated with one of three different trolamine/acetic acid buffers. A trolamine/acetic acid buffer concentrate was used to match the conductivity of the loads with that of the equilibration buffers. The column was challenged with each conditioned feed at a resin loading level of 200 g/L at 0.32 ml/min flow rate. After the loading, the columns were flushed with 20 CV of equilibration buffer. The flow-through and wash were collected and measured for protein concentrations by UV₂₈₀, aggregate and fragment levels by SEC method and HCP's by an enzyme linked immunoadsorbent assay (ELISA).

Table 3 summarizes the reduction of aggregate and fragment levels and HCP's upon flow-through polishing by Capto™ Adhere or Capto MMC™ resin at pH 7.8 under various conductivity conditions.

TABLE 3
Aggregates, fragments, HCP clearance, and yields for mAb3 by Capto ™
Adhere or Capto MMC ™ flow-through chromatography

| | | | Aggr. | Monomer | Frag | |
|---|---|---|---|---|---|---|
| Step | Loading Conditions | Yield (%) | (%) | (%) | (%) | HCP (ng/mg) |
| JSR Protein A | Clarified harvest, | 99.6 | 1.407 | 97.886 | 0.707 | 4818 |
| Capture | 4.5 g/L | | | | | |
| Low pH | pH 7.8, 3 mS/cm, | 95.8 | 1.380 | 97.400 | 0.680 | 2731 |
| Inactivation | 3.14 g/ml | | | | | |
| Emphaze | | | | | | |
| AEX depth | | | | | | |
| filter | | | | | | |
| Capto Adhere | pH 7.8, 3 mS/cm, | 86.0 | 0.469 | 99.531 | 0.000 | 1147 |
| | 200 mg/ml | | | | | |
| Capto Adhere | pH 7.8, 6 mS/cm, | 92.9 | 0.608 | 99.392 | 0.000 | 2175 |
| | 200 mg/ml | | | | | |
| Capto Adhere | pH 7.8, 7.8 mS/cm, | 95.2 | 0.859 | 98.989 | 0.152 | 2182 |
| | 200 mg/ml | | | | | |
| Capto MMC | pH 7.8, 3 mS/cm, | 87.1 | 0.552 | 99.072 | 0.376 | 621 |
| | 200 mg/ml | | | | | |

At pH 7.8, lower conductivities give the best removal of aggregates, fragments and HCP's. The Capto™ Adhere resin is better at removing antibody fragments. The Capto MMC™ resin is more efficient at reducing HCP levels than Capto™ Adhere resin. Higher conductivities gave higher yields at the expense of product quality.

### 4. Flow-Through Polishing of mAb3 by a Combination of Capto MMC™ and Capto™ Adhere Resin

The lot of mAb3 drug substance from the previous experiments was also used as the feed material for Capto MMC™/Capto™ Adhere combination flow-through processing. One ml Hitrap Capto MMC™ and Capto™ Adhere column were placed in a series and run as one operation (Capto MMC™ column followed by a Capto™ Adhere column) The columns, in series, were equilibrated with one of three different trolamine/acetic acid buffers. A trolamine/acetic acid buffer concentrate was used to match the conductivity of the loads with that of the equilibration buffers. The combined columns were challenged with each conditioned feed at a resin loading level from 187 to 600 g/L and at 0.32 ml/min flow rate. The flow-through and wash were collected and measured for protein concentrations by UV₂₈₀, aggregate and fragment levels by SEC method and HCP's by an enzyme linked immunoadsorbent assay (ELSIA).

Table 4 summarizes the reduction of aggregate and fragment levels and HCP's upon flow-through polishing by the combination of Capto™ Adhere and Capto MMC™ resins at pH 7.8 under various conductivity conditions.

TABLE 4
Aggregates-fragments-HCP clearance and yields for mAb3 by Capto
MMC ™ and Capto ™ Adhere combination flow-through
chromatography

| | Loading | Yield | Aggr. | Monomer | Frag. | HCP |
|---|---|---|---|---|---|---|
| Step | Conditions | (%) | (%) | (%) | (%) | (ng/mg) |
| Low pH | pH 7.8, | NA | 1.523 | 98.234 | 0.243 | 2657 |
| Inactivation | 3 mS/cm, | | | | | |
| Emphaze | 3.14 g/ml | | | | | |
| AEX depth | | | | | | |
| filter | | | | | | |
| Capto | pH 7.8, | 88.3 | 0.584 | 99.416 | 0.000 | 996 |
| MMC/Capto | 3 mS/cm, | | | | | |
| Adhere | 600 mg/ml | | | | | |
| Capto | pH 7.8, | 94.9 | 0.703 | 99.297 | 0.000 | 1525 |
| MMC/Capto | 4.5 mS/cm, | | | | | |
| Adhere | 600 mg/ml | | | | | |
| Capto | pH 7.8, | 86.7 | 0.366 | 99.634 | 0.000 | 1382 |
| MMC/Capto | 6 mS/cm, | | | | | |
| Adhere | 187 mg/ml | | | | | |
| Capto | pH 7.8, | 96.5 | 0.885 | 99.116 | 0.000 | 1726 |
| MMC/Capto | 6 mS/cm, | | | | | |
| Adhere | 600 mg/ml | | | | | |

At pH 7.8, lower conductivities give the best removal of aggregates, fragments and HCP's. Higher conductivities and higher loads gave higher yields at the expense of product quality.

### 5. Flow-Through Polishing of mAb3 by a Combination of Capto MMC™ and Capto™ Adhere Resin Using Material Derived from Different Protein a Capture Resins

Mab3, generated from a process using different Protein A capture resins (Amsphere™ Protein A (JSR Life Sciences) and MabSelect Sure™ —(GE Healthcare)) followed by anion exchange depth filter polishing, were used as the load materials for Capto MMC™/Capto™ Adhere combination flow-through processing. One ml HiTrap Capto MMC™ and Capto™ Adhere column were placed in a series and run as one operation (Capto MMC™ column followed by a Capto™ Adhere column) The polishing columns, in series, were equilibrated with a trolamine/acetic acid buffer pH 7.8 at 5 mS/cm conductivity. A trolamine/acetic acid buffer concentrate was used to match the conductivity of the loads with that of the equilibration buffers for the combined columns. The combined columns were challenged with each conditioned feed at a resin loading level from 187 to 600 g/L and at 0.32 ml/min flow rate. The flow-through and wash were collected and measured for protein concentrations by UV₂₈₀, aggregate and fragment levels by SEC method and HCP's by an enzyme linked immunoadsorbent assay (ELSIA).

Table 5 summarizes the reduction of mAb3 aggregate and fragment levels and HCP's upon flow-through polishing by the combination of Capto™ Adhere and Capto MMC™ resins at pH 7.8 at 5 mS/cm conductivity using load materials derived from different Protein A capture resins

TABLE 5
Aggregates, fragments, HCP clearance and yields for mAb3 by a combination
of Capto MMC ™ and Capto ™ Adhere polishing resins

| | JSR resin | | | | | MabSelect SuRe Resin | | | | |
|---|---|---|---|---|---|---|---|---|---|---|
| | | Purity % | | | HCP | | Purity % | | | HCP |
| Step | Yield % | aggreg. | monomer | frag. | ng/mg | Yield % | aggreg. | monomer | frag. | ng/mg |
| Primary | 96.4 | | | | NA | 96.4 | | | | NA |
| Recovery | | | | | | | | | | |
| Protein A | 91.8 | 0.860 | 98.731 | 0.409 | 1467 | 88.3 | 0.874 | 98.681 | 0.445 | 596 |
| capture | | | | | | | | | | |
| Low pH | 93.2 | 1.258 | 98.385 | 0.357 | 793 | 97.8 | 1.219 | 98.389 | 0.392 | 10 |
| Inact. | | | | | | | | | | |
| Emphaze | | | | | | | | | | |
| AEX depth | | | | | | | | | | |
| filter at pH | | | | | | | | | | |
| 7.8, 3 mS/cm | | | | | | | | | | |
| at | | | | | | | | | | |
| 1.675 g/ml | | | | | | | | | | |
| loading | | | | | | | | | | |
| Capto MMC/ | 91.9 | 0.293 | 99.707 | 0.000 | 230 | 91.4 | 0.122 | 99.878 | 0.000 | 7 |
| Capto | | | | | | | | | | |
| Adhere at | | | | | | | | | | |
| pH 7.8, 5 mS/cm | | | | | | | | | | |
| at | | | | | | | | | | |
| 200 mg/ml | | | | | | | | | | |
| loading | | | | | | | | | | |

Mab3 drug substance purified by Protein A capture using MabSelect SuRe™ resin resulted in HCP's level that were 66% lower than that when using the JSR Ampshere resin under identical processing conditions. Other quality attributes were similar between the two Protein A capture methods. Additional differences were seen in further processing through the Emphaze™ AEX depth filter. A 46% reduction of HCP was realized with the material produced by the Ampshere resin; whereas a 98% reduction in HCP was observed when the material was produced by the MabSelect SuRe™ resin. Processing of these two feed streams through the combination Capto MMC™/Capto™ Adhere combination further reduced the HCP levels. Single digit levels of HCP were achieved using material processed with the MabSelect SuRe™ resin. Both processes gave similar yields and reduction of antibody aggregates and fragments

### 6. Flow-Through Polishing of mAb3 by a Combination of Capto™ Adhere and Capto MMC™ Resin

Mab3, generated from a process using MabSelect SuRe™ Protein A resin followed by anion exchange depth filter polishing, was used as the load material for Capto™ Adhere and Capto MMC™ flow-through processing. Ten ml Capto™ Adhere and Capto MMC™ were packed for use. The mixed mode polishing columns were equilibrated with a trolamine/acetic acid buffer pH 7.8 at 4.5 or 5 mS/cm conductivity. A trolamine/acetic acid buffer concentrate was used to match the conductivity of the loads with that of the equilibration buffers for mixed mode columns. Load material at pH 7.8 4.5 mS/cm was first applied to the Capto™ Adhere column at 3.2 ml/min. Flow-through and wash material form the Capto™ Adhere column was then applied to the Capto MMC™ column at either 4.5 or 5 mS/cm conductivity at a flow rate of 3.2 ml/min. The flow-through and wash were collected. Samples were measured for protein concentrations by UV₂₈₀, aggregate and fragment levels by SEC method. HCP's and residual leached Protein A levels were measured with an enzyme linked immunoadsorbent assays (ELSIA).

TABLE 6
Aggregates, fragments, HCP and Residual Protein A clearance and yields for
mAb3 by a combination of Capto ™ Adhere and Capto MMC ™ polishing resins

| | yield | Aggreg. | Monomer | Frag. | HCP | Protein A |
|---|---|---|---|---|---|---|
| Step | (%) | (%) | (%) | (%) | (ng/mg) | (ng/mg) |
| MabSelect SuRe Protein A | 93.7 | 0.859 | 98.288 | 0.853 | 613 | 1.898 |
| capture eluate | | | | | | |
| Low pH Inact. Emphaze AEX | 94.9 | 1.068 | 98.133 | 0.800 | 13 | ND |
| depth filter FTW at pH 7.8, 4.5 mS | | | | | | |
| conductivity, 1 g/ml load | | | | | | |
| Capto Adhere at pH 7.8, 4.5 mS | 91.5 | 0.425 | 99.575 | 0.000 | 9 | ND |
| conductivity loaded at 200 mg/ml | | | | | | |
| Capto Adhere FTW applied to | 82.8 | 0.199 | 99.801 | 0.000 | 3 | 0.013 |
| Capto MMC at pH 7.8, 4.5 mS | | | | | | |
| conductivity at 200 mg/ml | | | | | | |
| Capto Adhere FTW applied to | 89.0 | 0.343 | 99.657 | 0.000 | 3 | <0.007 |
| Capto MMC at pH 7.8, 5 mS | | | | | | |
| conductivity at 200 mg/ml | | | | | | |

Mab3 was purified by a combination of Protein A capture (MabSelect SuRe™ resin) followed by AEX depth filtration, followed by Capto™ Adhere and Capto MMC™ chromatography. High purity and low levels of HCP's and residual Protein A were achieved with this streamlined process. Overall downstream recoveries up to 72% were achieved.

### 7. Flow-Through Polishing of mAb3 by Phenyl Sepharose HP Resin

Mab3 drug substance, generated from a process using MabSelect SuRe™ Protein A resin followed by anion exchange depth filter polishing, was used as the load material for Phenyl Sepharose HP flow-through processing. A 1 ml Hitrap Phenyl HP Sepharose column was equilibrated with one of four different trolamine/acetic acid/Na citrate buffers. A trolamine/acetic acid/Na citrate buffer concentrate was used to match the conductivity of the loads with that of the equilibration buffers. The Phenyl HP column was challenged with each conditioned feed at a resin loading level from 25 to 100 g/L and at 0.32 ml/min flow rate. The flow-through and wash were collected and measured for protein concentrations by UV₂₈₀, aggregate and fragment levels by SEC method and HCP's levels by an enzyme linked immunoadsorbent assay (ELSIA).

Table 7 summarizes the reduction of aggregate and fragment levels and HCP's upon flow-through polishing by Phenyl Sepharose HP resin at pH 7.5 with concentrations of 300 mM to 400 mM Na citrate in the load and buffers.

TABLE 7
Aggregates-fragments-HCP clearance and yields for mAb3 by a Phenyl
Sepharose HP polishing resin

| | | Yield | Aggr. | Monomer | Frag. | HCP |
|---|---|---|---|---|---|---|
| Step | Loading Conditions | (%) | (%) | (%) | (%) | (ng/mg) |
| Low pH Inactivation | pH 7.8, 3 mS/cm, | 97.8 | 1.223 | 98.447 | 0.330 | 10 |
| Emphaze AEX | 3.14 g/ml | | | | | |
| depth filter FTW | | | | | | |
| Phenyl HP 300 mM | pH 7.5, 300 mM Na | 93.1 | 0.000 | 99.730 | 0.270 | 2 |
| FTW | citrate, 25 mg/ml | | | | | |
| | load | | | | | |
| Phenyl HP 300 mM | pH 7.5, 300 mM Na | 96.1 | 0.244 | 99.447 | 0.309 | 9 |
| FTW | citrate, 50 mg/ml | | | | | |
| | load | | | | | |
| Phenyl HP 330 mM | pH 7.5, 330 mM Na | 93.2 | 0.000 | 99.725 | 0.275 | 2 |
| FTW | citrate, 25 mg/ml | | | | | |
| | load | | | | | |
| Phenyl HP 330 mM | pH 7.5, 330 mM Na | 95.4 | 0.000 | 99.693 | 0.307 | 3 |
| FTW | citrate, 50 mg/ml | | | | | |
| | load | | | | | |
| Phenyl HP 350 mM | pH 7.5, 350 mM Na | 91.2 | 0.046 | 99.501 | 0.453 | 1 |
| FTW | citrate, 25 mg/ml | | | | | |
| | load | | | | | |
| Phenyl HP 350 mM | pH 7.5, 350 mM Na | 94.6 | 0.232 | 99.234 | 0.535 | 2 |
| FTW | citrate, 50 mg/ml | | | | | |
| | load | | | | | |
| Phenyl HP 375 mM | pH 7.5, 375 mM Na | 92.7 | 0.288 | 99.199 | 0.513 | 2 |
| FTW | citrate, 100 mg/ml | | | | | |
| | load | | | | | |
| Phenyl HP 400 mM | pH 7.5, 400 mM Na | 90.0 | 0.000 | 99.749 | 0.251 | 2 |
| FTW | citrate, 100 mg/ml | | | | | |
| | load | | | | | |

The best conditions for removal of aggregates, fragments and HCP's were at lower loading levels and higher Na citrate concentrations. Lower Na citrate concentrations gave higher yields at the expense of product quality. Optimal buffer concentrations for product quality were in the range of 350 to 400 mM Na citrate. Higher Na citrate concentrations allowed for higher loading amounts, while maintaining adequate aggregate and HCP removal and yield recoveries. Removal of antibody fragments was not observed under the above run chromatography conditions.

### 8. Flow-Through Polishing of mAb3 by a Combination of Capto™ Adhere Resin and Phenyl Sepharose HP Resin

Mab3 drug substance, generated from a process using MabSelect SuRe™ Protein A resin followed by anion exchange depth filter polishing, was used as the load material for Phenyl Sepharose HP flow-through processing. A ten ml Phenyl Sepharose column was packed for use. A ten ml Capto™ Adhere polishing column was equilibrated with a trolamine/acetic acid buffer pH 7.8 at 4.5 mS/cm conductivity. Load material was first applied to the Capto™ Adhere column at 200 mg/ml at a flow rate of 3.2 ml/min. Flow-through material from the Capto™ Adhere column was diluted with 1.14 M Na citrate buffer concentrate to bring the material to a concentration of 350 mM Na citrate to match the Phenyl column running condition. The Phenyl HP column was challenged with conditioned feed at a resin loading level of 50 g/L at 3.2 ml/min flow rate. The flow-through and wash were collected. Samples were measured for protein concentrations by UV₂₈₀, aggregate and fragment levels by SEC method. HCP's and residual leached Protein A levels were measured with an enzyme linked immunoadsorbent assays (ELSIA).

TABLE 8
Aggregates, fragments and HCP clearance and yields for mAb3 by a
combination of Capto ™ Adhere and Phenyl Sepharose HP polishing resins

| | | Aggreg. | Monomer | Frag. | HCP | Protein A |
|---|---|---|---|---|---|---|
| Step | Yield (%) | (%) | (%) | (%) | (ng/mg) | (ng/mg) |
| MabSelect SuRe Protein A | 93.7 | 0.859 | 98.288 | 0.853 | 613 | 1.898 |
| capture | | | | | | |
| Low pH Inact. Emphaze | 94.9 | 1.068 | 98.133 | 0.800 | 13 | ND |
| AEX depth filter FTW at | | | | | | |
| pH 7.8, 4.5 mS/cm | | | | | | |
| conductivity, 1 g/ml | | | | | | |
| Capto Adhere FTW at pH | 91.5 | 0.425 | 99.575 | 0.000 | 9 | ND |
| 7.8, 4.5 mS/cm | | | | | | |
| conductivity, 200 mg/ml | | | | | | |
| load | | | | | | |
| Phenyl Sepharose HP FTW | 92.6 | 0.000 | 100.000 | 0.000 | 1 | <0.008 |
| at pH 7.5, 350 mM Na | | | | | | |
| citrate, 50 mg/ml load | | | | | | |

Mab3 was purified by a combination of Protein A capture (MabSelect SuRe™ resin) followed by AEX depth filtration, followed by Capto™ Adhere and Phenyl Sepharose HP (HIC) chromatography in flow-through modes. High purity and low levels of HCP's and residual Protein A were achieved with this streamlined process. Overall downstream recovery of 75% was achieved.

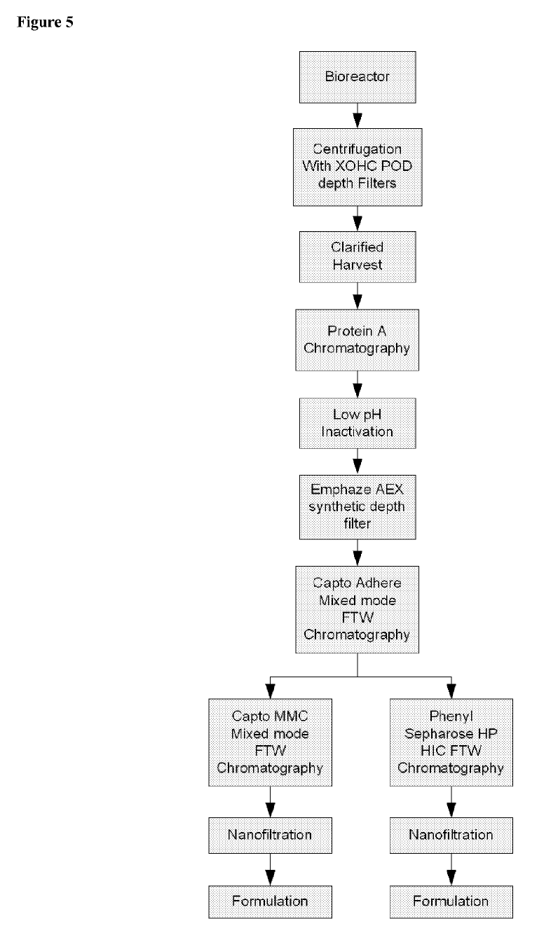

Two scenarios were investigated as alternative high throughput downstream processes, for the purification of mAb3. Both processes begin with capture of antibody by Protein A chromatography. MabSelect SuRe™ was found to be the better resin for achieving the final product quality needed. MabSelect SuRe™ was found to have similar binding capacity to that of the Amsphere resin. The Amphere resin could be operated at higher flow rates and had a lower resin cost. A synthetic depth filter was employed (3M Emphaze™ AEX hybrid purifier) to remove process impurities after low pH viral inactivation without introducing beta glucans (normally found in depth filter containing cellulose). The output from the capture operations could be further processed downstream by either a combination of Capto™ Adhere and Capto MMC™ mixed mode chromatography or Capto™ Adhere and Phenyl Sepharose HP hydrophobic interaction chromatography. Both processes produce bulk drug substance that meet or exceed product quality attributes of commercially produced antibodies. FIG. 5 depicts several flow schemes embodying features of the present method.

The mixed mode resins and hydrophobic interaction resins were explored for flow-through polishing of various mAbs. These resins can be used in combination with each other, or with other conventional chromatography methods to achieve desired protein separations. For instance, one exemplary process based on Protein A capture, Capto™ Adhere, and Capto MMC™ flow-through polishing demonstrated excellent product quality and high yield for different mAbs.

Various publications are cited herein, the contents of which are hereby incorporated by reference in their entireties.

What is claimed is:

1. A method for producing a impurity-reduced antibody preparation from a sample mixture comprising an antibody and at least one impurity, said method comprising:
(a) contacting said sample mixture to an affinity chromatography resin and collecting an affinity chromatography sample;
(b) filtering the sample through a depth filter to obtain a filtered affinity chromatography sample;
(c) contacting said filtered affinity chromatography sample to a resin having both ion exchange and hydrophobic interaction functionalities and collecting a final sample, wherein said final sample comprises said impurity-reduced antibody preparation.

2. The method of claim 1, wherein said resin of step (c) is a mixed mode resin.

3. The method of claim 1, wherein said ion exchange function of said resin of step (c) is cationic.

4. The method of claim 1, wherein said ion exchange function of said resin of step (c) is anionic.

5. The method of claim 1, wherein said contacting said filtered affinity chromatography sample of step (c) is performed in a flow-through mode.

6. The method of claim 3, wherein said resin of step (c) is selected from the group consisting of Capto adhere and Capto adhere ImpRes.

7. The method of claim 4, wherein said resin of step (c) is selected from the group consisting of Capto MMC, Capto MMC ImpRes, and Nuvia cPrime.

8. The method of claim 1, wherein one type of mixed mode resin is used in step (c).

9. The method of claim 8, wherein said final sample of step (c) is further processed by contacting said final sample to at least one additional mixed mode resin.

10. The method of claim 1, wherein more than one type of mixed mode resins are used in step (c).

11. The method of claim 10, wherein said more than one type of mixed mode resins are of a different charge.

12. The method of claim 11, wherein said more than one type of mixed mode resins are positioned to function in a tandem mode.

13. The method of claim 5, wherein said final sample is further processed by contacting the sample to a hydrophobic interaction chromatography (HIC) resin.

14. The method of claim 13, wherein said further processing is performed in a flow-through mode.

15. The method of claim 14, wherein said mixed mode resin of step (c) is Capto adhere.

16. The method of claim 14, wherein said HIC resin is selected from the group consisting of Phenyl Sepharose HP, Capto Phenyl, Phenyl Sepharose, and Toyopearl Phenyl resins.

17. The method of claim 1, wherein said depth filter is a synthetic depth filter.

18. The method of claim 17, the said synthetic depth filter is Emphaze AEX purifier.

19. The method of claim 1, wherein said impurities are host cell proteins (HCP), aggregates, fragments, and/or leached Protein A.

20. A composition of matter comprising an antibody preparation produced by the method of claim 1.
